(19) United States
(12) Patent Application Publication
(10) Pub. No.: US 2007/0166774 A1
(43) Pub. Date: Jul. 19, 2007

(54) Functional immunoassay

(76) Inventors: Ernest V. Groman, Brookline, MA (US); Christopher P. Reinhardt, Worcester, MA (US); Dennis E. Vaccaro, Wellesley, MA (US)

Correspondence Address: LAWSON & WEITZEN, LLP, 88 BLACK FALCON AVE, SUITE 345, BOSTON, MA, 02210, US

(21) Appl. No.: 11/633,909

(22) Filed: Dec. 5, 2006

Related U.S. Application Data
(60) Provisional application No. 60/759,368, filed on Jan. 17, 2006.

(51) Int. Cl. G01N 33/53; C07K 16/18

(52) U.S. Cl. 435/7.92; 530/388.1

(57) ABSTRACT

Compositions, methods, and functional immunoassays for measuring functioning of organs, receptors, and biological processes in a subject are provided. Functional immunoassay combines pharmacokinetics of xenobiotics after administering one or more of the compositions to a subject by quantifying the xenobiotic in samples taken from the subject. The method provides a xenobiotic that is processed principally by a single organ system with minimal metabolism and involves administering the xenobiotic to the subject, measuring its concentration in samples of biological material obtained over time, calculating a kinetic parameter that describes change in concentration over time, and associating this parameter with the functioning of the target organ. Functional immunoassay is used for measuring, for example, kidney, brain, lung, cardiovascular, gastrointestinal, and immune system function; progression of cancer and infectious disease; and diabetes and inflammatory disease status. Xenobiotics include, for example, drugs, ligands, hormone analogs, substrates, polysaccharides, and nucleic acids and respective analogs.

### RELATED APPLICATION

The present application claims the benefit of provisional application Ser. No. 60/759,368 filed in the U.S. Patent and Trademark Office on Jan. 17, 2006, which is hereby incorporated by reference herein in its entirety.

### TECHNICAL FIELD

The field relates to immunoassay methods that gauge functional performance of an organ or organ system by measuring kinetic behavior of an administered xenobiotic compound or plurality of xenobiotic compounds.

### BACKGROUND

The general technique of immunoassay, like so many other scientific discoveries, as developed as a result of some serendipitous events and careful observation. In the mid-1950s Solomon Berson and Rosalyn Yalow were studying the role of insulin in diabetics when they noted that the treated diabetics carried antibodies to the peptide hormone. They discovered that these antibodies could bind radioactively labeled insulin, and the science of immunoassay was born.

It was not long before other investigators discovered that immunoassay could be used to measure other molecules besides peptide hormones. Assays for the thyroid hormones, drugs, and many more compounds were devised. Their effectiveness in readily measuring substances that previously could be measured only by long and laborious bioassays or indirect chemical methods led to immunoassay becoming accepted by the biomedical community as a standard technique.

New immunoassays are added to the patent literature yearly. An immunoassay technique to measure ceruloplasmin concentrations in blood for the diagnosis of Wilson's disease, a copper metabolism defect wherein the body cannot efficiently excrete copper is shown in U.S. Pat. No. 6,806,044 (Hahn et al.). This method measures holoceruloplasmin concentration in a blood spot using a standard curve obtained by either an enzyme-linked immunosorbent assay (ELISA) or a dissociation-enhanced time-resolved fluoroimmunoassay employing a specific polyclonal antibody and a monoclonal antibody of holoceruloplasmin. Similar methods include an immunoassay to measure parathyroid hormone (PTH; U.S. Pat. No. 6,689,566, Cantor et al.); an immunoassay for hepatitis C (U.S. Pat. No. 6,596,476, Lesniewski et al.); an immunoassay to diagnosis osteoporosis (U.S. Pat. No. 6,258,552, Shiraki et al.); and an immunoassay to evaluate cardiac allograft rejection (U.S. Pat. No. 6,117,644, DeBold). These tests measure a naturally occurring biochemical within a subject, and an observed upward or downward regulation of the measured biochemical provides diagnostic information concerning the presence or absence of disease.

Other immunoassay tests measure a concentration of a foreign or non-endogenous chemical in biological fluids. For example, particular steroids are measured by immunoassay (U.S. Pat. No. 6,201,141, Williams et al.), such as during hormonal replacement therapy, or to determine steroid misuse. Other immunoassays show methods that measure a concentration of cocaine and cocaine metabolites in biological fluids (U.S. Pat. No. 5,747,352, Yan et al. and U.S. Pat. No. 4,207,307, Kaul et al.), or measure tetrahydrocannabinoids (THC) levels (U.S. Pat. No. 5,463,027, Wang et al.). These tests use a single measurement either to determine the presence or absence of a compound such as THC, or to determine therapeutic effectiveness of a compound, such as a steroid used in replacement therapy.

Conventional immunoassay tests are (i) static in nature, confirming the diagnosis of disease (Yes/No) or the presence of a foreign substance (Yes/No) with a single measurement and (ii) do not involve administering a non-peptide foreign substance (xenobiotic) to establish a diagnosis. There is a need for methods to measure dynamic physiological processes such as liver function, kidney function (glomerular filtration rate and effective renal blood flow), cancer status and so forth, i.e., a need for a dynamic assay that measures an administered xenobiotic as a function of time in a subject, to evaluate and gauge functional performance of an organ or organ system over time.

### SUMMARY OF THE EMBODIMENTS

An aspect of the invention provides a composition for determining glomerular filtration rate or effective renal blood flow, the composition has at least one antibody that binds at least one xenobiotic selected from the group of iohexol, iothalamate, ioversol, gadolinium-DTPA, gadolinium-DOTA, GdHP-D03A, chromium-EDTA, inulin, para-aminohippuric acid, iodohippurate, and mercaptoacetyltriglycine-rhenium (MAG₃-Re), so that determining glomerular filtrations rate or effective renal blood flow is performing an immunoassay with the antibody, thereby determining a concentration of the xenobiotic in at least one post-administration sample from a subject.

In related embodiments, the antibody is an antibody fragment. In another related embodiment the antibody is polyclonal. Alternatively, the antibody is monoclonal. Further, in a related embodiment, the at least one antibody is a mixture of two or more antibodies, for example, a mixture of a polyclonal antibody and a monoclonal antibody. The various antibodies are formulated to perform immunoassay, for example, the antibody is formulated as a solid, i.e., crystallized, or attached to a solid phase. Alternatively, the antibody is in solution.

Another embodiment provides a composition that includes a first antibody selected from the group of: anti-iohexol, anti-iothalamate, anti-ioversol, anti-gadolinium-DTPA, anti-gadolinium-DOTA, anti-GdHP-D03A, anti-chromium-EDTA, and anti-inulin; and a second antibody selected from the group of: anti-para-aminohippuric acid, anti-iodohippurate and anti-MAG₃-Re. A related embodiment provides a first immunoassay that has the first antibody that measures glomerular filtration rate, and provides a second immunoassay that has the second antibody that measures effective renal blood flow, and the composition is a reagent for the first immunoassay and the second immunoassay which are performed together in a single tube.

Yet another aspect of the invention herein provides an immunizing composition that has an antigen for producing antibodies to a xenobiotic selected from the group of iohexol, iothaiamate, ioversol, gadolinium-DTPA, gadolinium-DOTA, GdHP-D03A, chromium-EDTA, inulin, para-aminohippuric acid, iodohippurate, or MAG₃-Re.

Another embodiment herein provides a diagnostic kit for quantifying by functional immunoassay a physiological process selected from at least one of glomerular filtration rate, effective renal blood flow, filtration fraction, blood volume, intravascular space, and bile acid activity, the kit having a xenobiotic composition, an antibody for the xenobiotic, a detection probe, a container, and instructions for use. The various embodiments of the xenobiotic compositions, the antibodies that bind to the xenobiotics, and the methods are all envisioned as relating also to the embodiments of the kits.

Another embodiment provides a composition that has a first xenobiotic selected from the group of iohexol, iothalamate, ioversol, gadolinium-DTPA, gadolinium-DOTA, GdHP-D03A, chromium-EDTA, and inulin, and that has a second xenobiotic selected from the group of para-aminohippuric acid, iodohippurate and MAG₃-Re. An embodiment of this composition is an immunoassay standard. An alternative embodiment of this composition is a pharmaceutical, for example, the composition is formulated with a pharmaceutically acceptable buffer or with a pharmaceutically acceptable salt.

Another embodiment provides a composition that has at least two xenobiotics selected from the group of iohexol, iothalamate, ioversol, gadolinium-DTPA, gadolinium-DOTA, GdHP-D03A, chromium-EDTA, and inulin. This provides a method for determining glomerular filtration rates simultaneously using two independent xenobiotic agents, each of which measures glomerular filtration rate.

In yet another embodiment a series of identical physiological tests are performed in a temporal series by sequential administration of at least two xenobiotics selected from the group of iohexol, iothalamate, ioversol, gadolinium-DTPA, gadolinium-DOTA, GdHP-D03A, chromium-EDTA, and inulin.

Another featured embodiment of the invention provides a method that involves obtaining at least one physiological material from a subject, such that the subject is further administered a composition having at least one xenobiotic, and obtaining, between 1 minute and 5 days following administration of the xenobiotic, at least one post-administration sample of the physiological material from the subject; and determining a concentration of the xenobiotic in the at least one sample by at least one immunoassay technique; and calculating a rate of change from a rate formula of the xenobiotic in the post-administration sample of the physiological material and expressing quantitatively the performance of the physiological process from the rate data.

The quantitative expression is a score, measure, or number that evaluates the performance of the physiological process or processes in the subject, useful to evaluate the health or prepare a diagnosis or a prognosis of the subject. Since an embodiment of the invention is that a plurality of physiological processes can be evaluated simultaneously, from one or more physiological materials from the same subject, a profile of physiological functions can be quickly and economically established by the methods herein.

Accordingly, in various embodiments, the rate formula conforms to a one compartmental model, and the one compartmental model is mathematically expressed as

C₁(t)=C₁(0)e^{-kt}

in which C₁ is the concentration of the xenobiotic in the compartment at a point in time and k is the rate of the xenobiotic leaving or entering the compartment. Alternatively, the rate formula conforms to an at least one compartmental model.

In general, the physiological material is at least one sample, typically a sample of a biological material such as blood, urine, feces, cerebrospinal fluid, lymph, or tissue from for example, a biopsy, and the like. The at least one sample includes a plurality of samples. Samples generally are obtained at a preset point or predetermined points in time, i.e., a “measured” point or points. The measured point can relate to an initial point in time, such as the time of administering the xenobiotic, or can relate to a point in time that a first physiological material is obtained. Alternatively, the at least one or plurality of samples are obtained at one or more known or measured, i.e., points in time that are recorded or identified.

In general, the at least one compound is a “xenobiotic”, i.e., has a chemical structure that is exogenous in origin or foreign in origin with respect to the subject, in that it is not endogenously produced and is not commonly found in the subject at any appreciable amount. In various embodiments of the method, the xenobiotic is at least one compound from the group of iohexol, iothalamate, ioversol, gadolinium-DTPA, gadolinium-DOTA, GdHP-D03A, chromium-EDTA, inulin, para-aminohippuric acid, iodohippurate, mercaptoacetyltriglycine-rhenium (MAG₃-Re), colloids, dextran, dextran derivatives, bile acid derivatives and bile analogs, and albumin derivatives. The bile acid derivatives and analogs, and the albumin derivatives are exemplary xenobiotics because they are chemically different from bile acids and albumins that are naturally occurring within the subject. In an embodiment of the xenobiotic that is a colloid, the size of the diameter of the particle is in the range of 2 nanometers to 1000 nanometers.

In an embodiment of the invention, the physiological process measures a kidney function, for example, the kidney function is selected from the group of glomerular filtration rate, effective renal blood flow, filtration fraction, and glomerular sieving. Accordingly, the xenobiotic is a glomerular filtration rate marker; for example, the glomerular filtration rate marker is selected from the group of iohexol, iothalamate, ioversol, gadolinium-DTPA, gadolinium-DOTA, GdHP-D03A, chromium-EDTA, and inulin.

In related embodiments, the xenobiotic is excreted through the glomeruli of the kidney. Alternatively, the xenobiotic is an effective renal blood flow marker, for example, the effective renal blood flow marker is selected from the group of para-aminohippuric acid, iodohippurate and MAG₃-Re. Alternatively, the xenobiotic is excreted through the tubules of the kidney.

Alternatively, the method includes formulating the composition to contain a first xenobiotic for measuring the glomerular filtration rate of the kidney and a second xenobiotic for measuring the effective renal blood flow of the kidney. Thus, the first xenobiotic is selected from the group of the glomerular filtration rate markers iohexol, iothalamate, ioversol, gadolinium-DTPA, gadolinium-DOTA, GdHP-D03A, chromium-EDTA, and inulin, and the second xenobiotic is selected from the group of effective renal blood flow markers para-aminohippuric acid, iodohippurate and MAG₃-Re. Alternatively, the method includes at least one marker of glomerular filtration, which glomerular filtration marker is excreted through the glomeruli of the kidney, and includes at least one marker of effective renal blood flow, which marker of effective renal blood flow is excreted through the tubules of the kidney.

A related embodiment of the method of the invention provides that the glomerular filtration rate is expressed in ml/minutes and is calculated from the formula

glomerular filtration rate=UV/P

where U is the concentration of the glomerular filtration marker in urine having units of mg/ml, V is flow rate of urine formation expressed in units of ml/min and P is concentration of the glomerular filtration marker measured in blood or plasma sample and expressed in units of mg/ml. A related embodiment further includes quantifying the glomerular filtration rate from the concentration of the xenobiotic in at least one blood, serum or plasma sample that was obtained at one or more measured time points. Another related method further includes quantifying the glomerular filtration rate from the concentration of the xenobiotic in at least one blood, serum or plasma sample and at least one urine sample obtained at one or more measured time points. Yet another related method further includes quantifying the glomerular filtration rate from the concentration of the xenobiotic in at least one urine sample obtained at one or more measured time points.

A related method provided herein expresses the effective renal blood flow in ml/minutes and is calculated from the formula

effective renal blood flow=UV/P

where U is the concentration of the effective renal blood flow marker measured in urine having units of mg/ml, V is the flow rate of urine formation expressed in units of ml/min and P is the concentration of the effective renal blood flow marker measured in the blood or plasma sample and expressed in units of mg/ml. In a related method, quantifying the effective renal blood flow value is assaying the concentration of the xenobiotic in at least one blood or plasma sample that was obtained at one or more measured time points. In a related method, quantifying the effective renal blood flow is assaying the concentration of the xenobiotic in at least one blood or plasma sample and at least one urine sample that was obtained at one or more measured time points. In another related method, quantifying the effective renal blood flow is assaying the concentration of the xenobiotic in at least one urine sample that was obtained at one or more measured time points. The methods herein provide quantitative expression of performance in the subject of the physiological process, which in certain embodiments is determined to be abnormal. In alternative embodiments, the performance in the subject of the physiological process is normal.

Another embodiment of the methods provided herein involves at least one physiological process that measures a function of the circulatory system, for example, blood volume, plasma volume, and intravascular volume. In a related method, the xenobiotic is selected from the group of dextran, dextran derivatives, and derivatized albumin. In another related embodiment the administered composition includes a first xenobiotic for measuring plasma volume of the circulatory system and a second xenobiotic for measuring intravascular volume of the circulatory system. Thus, the first xenobiotic is selected from the group of plasma volume markers including dextran, reduced dextran, albumin; and the second xenobiotic is selected from the group of intravascular volume markers including colloids.

Another embodiment of the methods provided herein involves at least one physiological process that measures at least one function of liver, for example, the liver function is selected from asialoglycoprotein receptor activity, reticuloendothelial system (RES) activity, bile acid absorption, and bile acid elimination.

In general in the methods herein, the subject is a warm blooded animal, for example, a mammal, for example a human, and the methods are also suitable for veterinary use such as for farm animals, research animals, and high value animals in captivity such as in zoos, and also for cold-blooded animals such as reptiles and fish. Although the general embodiments are envisioned for in vivo use, such as in whole animals, in another embodiment the subject is an isolated organ system.

In an embodiment of the invention, administering the xenobiotic is administering a plurality of doses over a dosage regimen for determining sequential performance of at least one physiological process, i.e., sequential performance involves quantifying performance over a period of time by administering at least one dose, or two or more does, the subsequent dose of the xenobiotic involving a re-iteration or a subsequent iteration of the methods herein.

In general, the immunoassay technique in various embodiments of the methods herein includes using at least one reagent selected from the group of an antibody, a binding protein, or a peptide. In embodiments in which a plurality of reagents is used, these may be used in combination or in separate iterations of the technique, as is convenient to the practitioner. The immunoassay technique involves at least one antibody that binds at least one xenobiotic selected from the group comprising anti-iohexol, anti-iothalamate, anti-ioversol, anti-gadolinium-DTPA, anti-gadolinium-DOTA, anti-GdHP-D03A, anti-chromium-EDTA, anti-inulin, anti-para-aminohippuric acid, anti-iodohippurate, anti- MAG₃-Re, anti-colloids, anti-dextran, anti-dextran derivatives, and anti-albumin derivatives.

In related embodiments of the method, the antibody is an antibody fragment. In related embodiments of the method, the antibody is polyclonal. In an alternative embodiment, the antibody is monoclonal. The phrase the “at least one antibody” in certain embodiments is a mixture of two or more antibodies, and in related embodiments, is a polyclonal antibody, and the other is a monoclonal antibody. In an alternative embodiment, the immunoassay technique includes at least one binding protein or peptide that binds the xenobiotic, which is selected from the group of iohexol, iothalamate, ioversol, gadolinium-DTPA, gadolinium-DOTA, GdHP-D03A, chromium-EDTA, inulin, para-aminohippuric acid, iodohippurate, MAG₃-Re, colloids, dextran, dextran derivatives, and albumin derivatives. In a related embodiment, the immunoassay technique includes a first antibody that binds a first xenobiotic selected from the group of iohexol, iothalamate, ioversol, gadolinium-DTPA, gadolinium-DOTA, GdHP-D03A, chromium-EDTA, and inulin; and includes a second antibody that binds a second xenobiotic selected from the group of para-aminohippuric acid, iodohippurate and MAG₃-Re. In an embodiment of the methods herein, the xenobiotic is approved for human use. In related embodiments, the xenobiotic is approved for veterinary use. In various alternative embodiments, the immunoassay technique involves a radio immunoassay (RIA), an enzyme immunoassay (EIA), an enzyme-linked immunosorbent assay (ELISA), a fluorescence immunoassay, an automated immunoassay, or a luminescent immunoassay or the like or combinations of these techniques.

A featured embodiment of the invention provides a method for quantifying at least one physiological process, the method involving: administering to the subject at least one xenobiotic, and obtaining between 1 minute and 5 days following administration of the xenobiotic at least one post-administration sample of at least one physiological material from the subject; determining a concentration of the xenobiotic in the sample by at least one immunoassay technique; and calculating a rate of change from a rate formula of the xenobiotic in the post-administration sample of the physiological material and expressing quantitatively the performance of the physiological process from the rate data. It is envisioned that all of the related embodiments described above with respect to the xenobiotics, physiological processes, antibodies, and methods, are related also to this particular method.

### BRIEF DESCRIPTION OF THE DRAWINGS

FIG. 1 is a set of graphs showing comparison of three different methods to measure glomerular filtration rate. FIG. 1, panel A is a comparison of the radioactive method to the neutron activation method, and shows that these methods are linearly correlated over the ranges of the xenobiotics on the axes. FIG. 1, panel B is a comparison of the neutron activation method to the functional immunoassay method, and shows that these methods are linearly correlated over the ranges of the xenobiotics on the axes.

FIG. 2 is a set of graphs showing comparison of the three different methods of measuring effective renal blood flow. FIG. 2, panel A is a comparison of the radioactive method to the neutron activation method, and shows that these methods are linearly correlated over the ranges of the xenobiotics on the axes. FIG. 2, panel B is a comparison of the neutron activation method to the functional immunoassay method, and shows that these methods are linearly correlated over the ranges of the xenobiotics on the axes.

FIG. 3 is a graph showing a comparison of two different methods of measuring filtration fraction of the kidney. The functional immunoassay method is directly compared to traditional radioactive method, and the two methods are shown to be linearly correlated over the ranges shown on the axes.

### DETAILED DESCRIPTION

Functional immunoassay provided herein is contrasted to conventional immunoassay by the following criteria: (i) functional immunoassay is dynamic and diverges from the conventional paradigm presented above by designing tests to evaluate and gauge the functional performance of an organ or organ system over time and, thereby typically uses multiple measurements of an analyte as a function of time and (ii) functional immunoassay depends upon administration of a xenobiotic to assess organ performance. Functional immunoassay achieves these objectives by utilizing immunoassay techniques as the readouts to measure the kinetic behavior of an administered xenobiotic or set of xenobiotics. The rates of clearance or of accumulation of the xenobiotic as measured in biological material, such as blood, urine and feces, provide the input parameters to calculate a functional index for the organ-of-interest.

TABLE 1
Comparison of Conventional Clinical Assays and Functional Immunoassay

| | Conventional Clinical | Functional |
|---|---|---|
| Characteristic | Immunoassay | Immunoassay |
| Single measurement | Yes | No |
| Multiple measurements | No | Yes |
| Injection of xenobiotic or | No | Yes |
| test probe required to | | |
| perform test | | |
| Measurement of naturally | Yes | No |
| occurring compound | | |
| Based on a rate of change of | No | Yes |
| test probe in physiological | | |
| material | | |
| Measures kidney function in | No | Yes |
| vivo and ex vivo | | |
| Measures receptor function | No | Yes |
| in vivo and ex vivo | | |

Functional immunoassays can be applied to measuring physiological processes including liver function, kidney function, cancer status and so forth. Exemplary functions to be measured by functional immunoassays as applied to kidney function include: glomerular. filtration rate and effective renal blood flow. Glomerular filtration rate is measured by injecting a compound into the body and determining over time a disappearance of the compound from the blood and a simultaneous appearance of the compound in the urine. By suitable measurement of this compound over time a value of the rate of its excretion can be calculated.

An ideal agent for measuring glomerular filtration rate possesses several of the following properties. (1) The compound is a xenobiotic, that is the ideal agent is a compound that does not occur naturally in the body. This property allows detection of the compound independent of endogenous compounds found in the body that could confuse measurement in blood or urine. Compounds that have similar structures to those preexisting in the body of the subject are naturally occurring peptides and their peptide analogs. Peptides for example are not ideal agents for this reason and use of peptides or other biologically derived compounds could lead to ambiguous measurements, therefore peptides are excluded from the class of xenobiotics used for measuring glomerular filtration rate. (2) The compound is structurally distinct from existing compounds found in the body. Examples of compounds having this property are Gadolinium DTPA and Gadolinium DOTA. Compounds lacking this property include peptides, especially peptides having an amino acid sequence of fewer than 12 amino acids. (3) The compound should result in no physiological changes following administration to the subject. Changes in functions such as blood pressure, diuresis, or urisis caused by a compound to measure glomerular filtration rate clearly would be counter-indicated due to its affecting the very parameter it is being used to measure. For this reason, naturally occurring molecular peptides that can have pharmacological properties, especially peptides that are members of the class known as ACE inhibitors, are excluded from the class of ideal compounds used to measure glomerular filtration rate. Compounds provided herein such as Gadolinium DTPA, Gadolinium DOTA and inulin, have no effect on physiological processes and therefore are suitable for measuring glomerular filtration. (4) The compound should remain as a free form in vivo, that is, not be bound following administration by serum proteins, by cells or by other structures within the body of the subject. Compounds such as Gadolinium DTPA, Gadolinium DOTA and inulin are examples of compounds that remain free and are not bound by such proteins, cells or organelles. Peptides and other naturally occurring compounds are, conversely, generally bound by proteins, cells, and organelles. (5) The compound must be stable and not subject to metabolism while inside the body. Compounds that undergo metabolism, conjugation or other chemical changes are difficult to measure because they are subject to the action of multiple physiological processes inducing enzymatic alterations including hydrolysis, acetylation, amination, glycosylation, sulfation, phosphorylation and other metabolic alterations. Gadolinium DTPA, Gadolinium DOTA and inulin in contrast are inert and are not subject to enzymatic metabolism. (6) The compound exhibits first pass excretion through the glomerulus of the kidney. This property is satisfied by Gadolinium DTPA, Gadolinium DOTA and inulin but not by peptides because they associate with binding proteins and other structures found within the body of the subjects. (7) The compound is not reabsorbed through the tubules of the kidney. This property is satisfied by Gadolinium DTPA, Gadolinium DOTA and inulin, however peptides fail this criterion because they are generally reabsorbed for recycling. For these reasons, peptides are excluded from compounds of the class of xenobiotics herein suitable for measuring glomerular filtration rate.

Two classes of reagents are used for performance of functional immunoassay technology for measuring glomerular filtration rate, each at a different point in the assay. One class of reagent is the set of injectable test reagents discussed above, for example, Gadolinium DTPA, Gadolinium DOTA and inulin. The other class of reagent includes antibodies, either polyclonal or monoclonal, for measuring the binding to the test reagent, or alternative binding agents. For Gadolinium DTPA and Gadolinium DOTA, numerous examples of antisera that specifically recognize and bind to these compounds are reported, however these antibodies have not been described for the measurement of Gadolinium DTPA and Gadolinium DOTA or for their use in the functional immunoassay technology, provided, and described below herein.

Two phase radioimmunotherapy based on bispecific monoclonal antibodies in which one arm recognizes a tumor antigen and the other a radiolabeled chelate is shown in Bosslet et al. 1991, Br J Cancer 63(5):681-686. Hybridomas were generated producing high avidity anti-metal chelate monoclonal antibodies (anti DTPA-Yttrium) that were fused with monoclonal antibodies specific for CEA or GIT mucin. This antibody is a bispecific conjugate formulated as an injectable for therapeutic applications.

Bispecific F(ab′)₂ antibodies from chimeric PAM4Fab′ and murine 734 (anti-iridium-DTPA) Fab′ fragments for therapeutic purposes are shown in Cardillo et al. 2004, Clin Cancer Res 10(10): 3552-61. This reference describes biodistribution studies and imaging properties of the radiolabelded bispecific antibodies.

Monoclonal antibodies that bind to water soluble complexes such as EDTA or DTPA are shown in U.S. Pat. No. 5,907,034 (Bosslet et al.), the monoclonal antibodies being coupled to filters or other supports for removing toxic heavy metals. Also provided is an immunoassay for the quantitative determination of EDTA or DTPA in aqueous solutions.

The use of ACE resistant N-acetyl-Ser-Asp-Lys-Pro analogues for preparing a reagent or marker adapted to measure a glomerular filtration rate is shown in PCT patent (WO 2004/096292 A2).

The isolation of a monoclonal anti DOTA-Yttrium antibody is shown in Song et al. 2003, Xi Bao Yu Fen Zi Mian Yi Xue Za Zhi 19(5): 476-479. This reference shows constructing an immune Fab phage antibody library.

The words below shall have the following meanings herein and in the claims, unless otherwise required by the context.

The word “antibody” refers to an antibody, an antiserum, an antibody fragment, a polyclonal antibody, a monoclonal antibody, a peptide, a protein, or other biochemical, that binds to an antigen or hapten and is used in the assay of a compound or xenobiotic.

The word “colloid” refers to any macromolecule or particle having a size less than about 1000 nm in diameter, or less than about 500 nm.

The phrase “detection probe” refers to one or more components of an immunoassay that generates the detected signal

The abbreviation “DOTA” refers to 1,4,7,10 tetraazacyclododecane-1,4,7,10-tetraacetic acid. When combined with gadolinium as an MRI contrast agent, it is called DOTAREM® (gadoterate meglumine).

The abbreviation “DTPA” refers to diethylenetetraaminepentacetic acid. When combined with gadolinium as an MRI contrast agent, it is called MAGNEVIST® (gadopentetate dimeglumine).

The abbreviation “EDTA” refers to ethylenediaminetetraacetic acid.

The phrase “functional immunoassay” refers to a technique that measures the functioning of organs, receptors, and biological processes in a subject by combining the pharmacokinetics of one or more xenobiotic chemicals after administering, for example, by injection in a subject, with the quantification of the xenobiotic (and its metabolites) by immunoassay. Applications of functional immunoassay include measurement of function of kidney, brain, lung, cardiovascular, gastrointestinal, and immune system; cancer and infectious disease detection, and diabetes and inflammatory disease status.

The phrase “effective renal blood flow” (ERBF) refers to a functional index of the kidney and is used to evaluate perfusion of renal tissue and tubular excretion rate.

The phrase “filtration fraction” (FF) refers to the volume of filtrate that forms from a given volume of plasma entering the glomeruli of the kidney.

The phrase “glomerular filtration rate” (GFR) refers to a functional index of the kidney and is used to evaluate how effectively materials are filtered from the body. GFR is defined as the volume of glomerular urine filtered per unit time.

The phrase “glomerular sieving” refers to a physical assessment of the integrity of the glomerular filtration section of the kidney and is measured by administering to a subject of a set of markers of increasing sized followed by assay of these markers in the blood and/or urine as a function of time.

The abbreviation “Gd-HP-DO3A” refers to gadoteridol also know as PROHANCE® (gadoteridol) and consists of a nonionic complex used as a paramagnetic MR contrast agent.

The abbreviation “HRP” refers to the enzyme horse radish peroxidase.

The word “immunoassay” refers to a laboratory technique that makes use of the binding between an antigen and its homologous antibody in order to identify and quantify the specific antigen or antibody epitope in a sample.

The phrase “immunogenic marker” refers to a xenobiotic that is immunologically distinct from endogenous compounds of the subject.

The phrase “intravascular space” refers to a functional index of the circulatory system and this index models the measurement obtained by tracking labeled red blood cells. Intravascular space can be expressed in units of ml/kg.

The abbreviation “MAG₃-Re” refers to mercaptoacetyltriglycine-rhenium.

Examples of “pharmaceutical formulations” or “pharmaceutically acceptable salts” or “pharmaceutically acceptable buffers” for pharmacological and biomedical use include but are not limited to applications of functional immunoassay used in the in vivo phase requiring administration of test sample to the subject or in the immunoassay phase of functional immunoassay. Typical buffers have a pH range between 4 and 10 and a salt concentration between 0.001 and 1 M.

The abbreviation “PBS” refers to phosphate buffered saline, pH 7.4.

The phrase “physiological material” or “physiological fluid” refers to, for example, blood, plasma, serum, urine, feces, saliva, bile, cerebrospinal fluid, lymph, and any tissue from a biopsy or other source of tissue such as tissue culture or artificial organ system, and the like.

The phrase “physiological function” refers to the concept of homeostasis whereby cells, organs, and organ systems act to maintain a static or constant condition in the internal environment of an organism. When organs function normally, homeostasis is maintained. When organs function abnormally or deteriorate, homeostasis is not maintained or is threatened. The acts of biological and biochemical activities that cells, organs, and organ systems perform to maintain homeostasis is defined as the physiological function of that cell or organ system.

The phrase “physiological process” refers to the specific operations that cells, organs, and organ systems do to perform to their physiological functions.

The phrase “physiological sample” refers to a portion of a physiological fluid or physiological material, such as a portion of a specimen of blood, plasma, serum, urine, saliva, bile, feces cerebrospinal fluid, lymph, tissue biopsy, and the like.

The phrase “plasma volume” refers to a functional index of the circulatory system and can be expressed in units of ml/kg.

The phrase “renal sieving” refers to a general process of the kidneys that selects materials of different sizes that pass from the blood to the urine, while retaining other materials in the blood.

The phrase “sample material” refers to physiological specimen collected from an animal or man at a measured or identified time. A sample refers to a portion of a physiological material.

The word “subject” refers to a person, non-human animal or other organism, subject includes a warm-blooded animal that is a mammal or a bird, including human, for example, an animal of agricultural or zoological importance. Other examples of “subjects” include pets, both warm blooded and cold blooded, show animals such as horses, cats and dogs, and sport related animals including race horses.

The phrase “test reagent” refers to a xenobiotic that is administered to a subject, after which the kinetic behavior of the xenobiotic in the subject is used to provide diagnostic information concerning the functional performance of an organ or organ system.

The phrase “total blood volume” is a functional index of the circulatory system and is the summation of the plasma volume (extracellular fluid and the hematocrit).

The phrase “xenobiotic” or “test probe” refers to a chemical that is not a natural component of the organism exposed to it. Examples of xenobiotics include drugs, ligands, hormone analogs, substrates, proteins and protein derivatives, polysaccharides and polysaccharide derivatives, and nucleic acids and nucleic acid derivatives. Specifically excluded from the class of compounds called xenobiotics with respect to application of the measurement of glomerular filtration rate herein are natural peptides and their peptide analogs. A subclass of peptides and peptide analogs are those compounds that bind to proteins, cells, or other bodies in the test organism and those compounds that have pharmacological properties and that affect physiological processes. Examples of peptides that have pharmacological properties are peptides that are angiotensin converting enzyme (ACE) inhibitors.

An embodiment of the invention is a method of utilizing immunoassay techniques to measure the kinetic behavior of an administered test reagent or set of test regents for the purpose of gauging the functional performance of an organ or organ system.

Another embodiment of the invention is a method to measure glomerular filtration rate (GFR) of the kidney by utilizing immunoassay techniques to measure the kinetic clearance of an administered filtration reagent from the body. An immunoassay technique is used to measure the concentration of the reagent in collected blood and urine samples. The concentration measurements are then imported into a kinetic model to obtain the GFR value.

Another embodiment of the invention is a method to measure effective renal blood flow (ERBF) of the kidney by utilizing immunoassay techniques to measure the kinetic clearance of an administered tubular filtration reagent from the body. An immunoassay technique is used to measure the concentration of the reagent in collected blood and urine samples. The concentration measurements are then imported into a kinetic model to obtain the ERBF value.

Yet another embodiment of the invention is a method to measure the filtration fraction (FF) of the kidney by utilizing immunoassay techniques to measure the kinetic clearance of an administered reagent or set of reagents from the body. One component of the composition or reagent or set of reagents is a xenobiotic that is a filtration marker and the other component is a xenobiotic that is a tubular filtration marker. An immunoassay technique is used to measure the concentration of the two marker xenobiotics in collected blood and urine samples. The concentration measurements are then imported into a kinetic model to obtain the FF value. As a result, another embodiment of the invention is the simultaneous measurement of three functional parameters of the kidney, i.e., GFR, ERBF and FF.

Another embodiment of the invention is a method to measure the plasma volume (PV) of the circulatory system by utilizing immunoassay techniques to measure the kinetic distribution of an administered reagent within the circulatory system of the body. An immunoassay technique is used to measure the concentration of the reagent, such as dextran, in collected blood samples. The concentration measurements are then imported into a kinetic model to obtain the plasma volume.

Another embodiment of the invention is a method to measure the intravascular space of the circulatory system by utilizing immunoassay techniques to measure the kinetic distribution of an administered reagent within the circulatory system of the body. An immunoassay technique is used to measure the concentration of the reagent, such as a colloid, in collected blood samples. The concentration measurements are then imported into a kinetic model to obtain the volume of the intravascular space of the circulatory system.

Yet another embodiment of the invention is a method to measure the total blood volume (TBV) of the circulatory system of the body by utilizing immunoassay techniques to measure the kinetic distribution of an administered reagent or set of reagents. One component of the reagent or set of reagents is a plasma marker and the other component is a marker of the intravascular space. An immunoassay technique is used to measure the concentration of the two markers in collected blood samples. The concentration measurements are then imported into a kinetic model to obtain the TBV value. As a result, another embodiment of the invention is the simultaneous measurement of three functional parameters of the circulatory system of the body, i.e., PV, the intravascular space and TBV.

Another embodiment of the invention is a method to measure the binding capacity and/or receptor response of the organ system, such as the hepatic system, by utilizing immunoassay techniques to measure the kinetic clearance or accumulation of an administered reagent in collected biological samples.

Yet another embodiment of the invention is a method to simultaneously measure the binding capacities and/or receptor responses of an organ system, such as the hepatic system, by utilizing immunoassay techniques to measure the kinetic clearance or accumulation of multiple, i.e., a plurality of administered reagents in collected biological samples.

Still another embodiment of the invention is a method to measure the binding capacity and/or receptor response of the organ system, such as the hepatic system, by utilizing immunoassay techniques to measure the kinetic clearance or accumulation of an administered reagent and to simultaneously measure the resulting clearance or accumulation of a metabolite of the reagent generated by the system in collected biological samples.

Another embodiment of the invention is a functional immunoassay test kit to measure at least one functional parameter of the organ of organ system, for example, to measure the glomerular filtration rate of the kidney; to measure the effective renal blood flow of the kidney; to measure the glomerular filtration rate and effective renal blood flow; to measure the filtration fraction of the kidney; to measure the plasma volume of the circulatory system; to measure the intravascular space of the circulatory system; to simultaneously measure the plasma volume and intravascular space of the circulatory system; to measure the total blood volume of the circulatory system; to measure the binding capacity of a receptor of an organ or organ system; and to measure the binding capacity of a receptor and to track its resulting metabolite of an organ or organ system.

### Antibodies

The present invention uses antibodies including isolated and/or purified antibodies that bind specifically to a target xenobiotic or molecule as defined herein for functional immunoassay. In certain embodiments, the antibodies of the invention are derived from particular heavy and light chain sequences and/or comprise particular structural features such as CDR regions comprising particular amino acid sequences. The invention provides isolated antibodies, methods of making such antibodies, and various additional compositions containing the antibodies, such as immunoconjugates and bispecific molecules comprising such antibodies. The invention also relates to methods of using the antibodies in vitro to determine a rate of at least one physiological process by binding to at least one xenobiotic target compound, thereby facilitating a diagnosis associated with a disorder or condition associated with the physiological process that may be abnormal, for example, hypernormal or hyponormal, and thereby distinguishing the amount or rate as a function of time, of the process in the subject, from that same process as is found in a normal subject.

In order that the present invention may be more readily understood, certain terms are first defined. Additional definitions are set forth throughout the detailed description.

The term “immune response” refers to the action of, for example, lymphocytes, antigen presenting cells, phagocytic cells, granulocytes, and soluble macromolecules produced by the above cells or the liver (including antibodies, cytokines, and complement) that results in the production of soluble immunoglobulins.

The term antibody as used to herein includes without limitation whole antibodies and any antigen binding fragment (i.e., “antigen-binding portion”) or single chains thereof. A naturally occurring antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., the target xenobiotics defined herein that are administered to a subject for analyzing rate of a physiological process). The antigen-binding function of an antibody can be performed by fragments of a full-length antibody. Examples of binding fragments encompassed within the term “antigen-binding portion” of an antibody include a Fab fragment, a monovalent fragment comprising the V_{L}, V_{H}, C_{L} and C_{H} domains; a F(ab)₂ fragment, a bivalent fragment comprising two Fab fragments linked by a disulfide bridge at the hinge region; a Fd fragment comprising the V_{H} and CH1 domains; a Fv fragment comprising the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds a target xenobiotic is substantially free of antibodies that specifically bind antigens other than this xenobiotic). An isolated antibody that specifically binds a target xenobiotic may, however, have cross-reactivity to other antigens, such as target molecules having the same or similar R groups. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “monoclonal antibody” refers to an antibody preparation in which each of the antibody protein molecules are substantially identical in amino acid sequence and share a single binding specificity and affinity. In a human monoclonal antibody, the protein molecules having variable regions in which both the framework and CDR regions are derived from human sequences. In one embodiment, the monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

As used herein, “isotype” refers to the antibody class (e.g., IgM, IgE, IgG such as IgG1 or IgG4) that is provided by the heavy chain constant region genes.

The phrases “an antibody recognizing a xenobiotic” and “an antibody specific for a xenobiotic ” are used interchangeably herein with the term “an antibody which binds specifically to a xenobiotic.”

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

Standard assays to evaluate the binding ability of the antibodies toward target xenobiotics are known in the art, including for example, ELISAs, western blots and radio-immune assays (RIAs). Suitable assays for ELISAs are described in detail in the Examples, however other immunoassay procedures are known to those skilled in the art. The binding kinetics (e.g., binding affinity) of the antibodies also can be assessed by standard assays known in the art, such as by Biacore analysis. Assays using antibodies are directed in this invention to evaluating the kinetics of target xenobiotics following passage through and/or action by target organs and organ systems etc. The functional properties (e.g., rate of appearance and excretion of the target xenobiotics in various organs and tissues) of the organs and/or organ systems can thereby be determined. The application and use of the antibodies in evaluating the functional properties of the organ and organ systems are described in further detail in the Examples.

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes.

An animal system for preparing hybridomas is exemplified by the murine system, and hybridoma production in the mouse system is a well-established procedure. Immunization protocols and techniques for isolation of immunized splenocytes for fusion are known in the art. Fusion partners (e.g., murine myeloma cells) and fusion procedures are also known.

In certain embodiments, the antibodies of the invention are monoclonal antibodies. Such monoclonal antibodies directed against one or more target xenobiotics can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

Monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Monoclonal antibodies of the invention can also be prepared using SCID mice into which human immune cells have been used to reconstitute and immune system, such that a human antibody response can be generated upon immunization of the mice. Such mice are described in, for example, U.S. Pat. Nos. 5,476,996 and 5,698,767 to Wilson et al.

### Pharmaceutical Compositions

In another aspect, the present invention provides a composition, e.g., a pharmaceutical composition, containing one or a combination of xenobiotics for administration to a subject. Such compositions may include one or a combination of (e.g., two or more different) xenobiotics, or xenobiotics in combination. For example, a pharmaceutical composition of the invention can comprise a combination of xenobiotics that have complementary activities for measuring physiological functions.

Pharmaceutical compositions of the invention also can be administered in combination, or can be combined with other types of agents. For example, the combination can include a xenobiotic of the present invention combined with at least one other agent such as an anti-inflammatory or antibiotic agent.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active xenobiotic, i.e., may be coated in a material to protect the xenobiotic from the action of acids and other natural conditions that may inactivate the xenobiotic.

The xenobiotics of the invention may include one or more pharmaceutically acceptable salts. A “pharmaceutically acceptable salt” refers to a salt that retains the desired biological activity of the parent xenobiotic and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediaminetetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active xenobiotic, use thereof in the pharmaceutical compositions of the invention is contemplated. Supplementary active xenobiotics can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin. However is most cases in the present invention it is likely that absorption delay is not desired, as preset or measured time points are used for determining kinetics of entry and/or excretion by a tissue or organ of the xenobiotic.

Sterile injectable solutions can be prepared by incorporating the active xenobiotic in the required amount in an appropriate solvent with one or a combination of ingredients enumerated above, as required, followed by sterilization microfiltration. Generally, dispersions are prepared by incorporating the active xenobiotic into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active xenobiotic that can be combined with a carrier material to produce a single dosage form will vary depending upon the subject being treated, and the particular mode of administration. The amount of the xenobiotic that can be combined with a carrier material to produce a single dosage form will generally be that amount of the composition which produces a desired concentration in the physiological organ or tissue being analyzed. Generally, out of one hundred percent, this amount will range from about 0.01 per cent to about ninety-nine percent of active ingredient, from about 0.1 per cent to about 70 per cent, or from about 1 percent to about 30 percent of active ingredient in combination with a pharmaceutically acceptable carrier.

Dosage regimens are adjusted to provide the optimum desired response (e.g., a response which is a physiologically measurable concentration in a biological fluid). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active xenobiotic calculated to produce the desired effect in association with the required pharmaceutical carrier. The specification for the dosage unit forms of the invention are dictated by and directly dependent on the unique characteristics of the active xenobiotic and the particular physiological concentration to be achieved, and the limitations in the art of compounding such an active xenobiotic for the treatment of sensitivity in individuals.

For administration of the xenobiotic, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg.

A composition or xenobiotic of the present invention can be administered by one or more routes of administration using one or more of a variety of methods known in the art. As will be appreciated by the skilled artisan, the route and/or mode of administration will vary depending upon the desired results. Routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Alternatively, a xenobiotic of the invention can be administered by a nonparenteral route, such as a topical, epidermal or mucosal route of administration, for example, intranasally, orally, vaginally, rectally, sublingually or topically. In most cases herein, the xenobiotic is administered by bolus injection or oral administration.

The active xenobiotics can be prepared with carriers that will protect the xenobiotic against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Compositions can be administered with medical devices known in the art. For example, in one embodiment, a composition of the invention can be administered with a needleless hypodermic injection device, such as the devices shown in U.S. Pat. Nos. 5,399,163; 5,383,851; 5,312,335; 5,064,413; 4,941,880; 4,790,824 or 4,596,556.

### Uses and Methods of the Invention

The antibodies of the present invention have in vitro utilities. In one embodiment, the antibodies of the invention can be used to detect levels of the target xenobiotic or xenobiotics. This can be achieved, for example, by contacting a sample (such as an in vitro sample) and a control sample with the anti-xenobiotic antibody under conditions that allow for the formation of a complex between the antibody and the target xenobiotic. Any complexes formed between the antibody and the target are detected and compared in the sample and the control. For example, standard detection methods, well known in the art, such as ELISA, can be performed using the compositions of the invention.

Accordingly, in one aspect, the invention further provides methods for detecting the presence of the target xenobiotic(s) in a sample, or measuring the amount of xenobiotic, comprising contacting the sample, and a control sample, with an antibody of the invention, or an antigen binding portion thereof, which specifically binds to the target, under conditions that allow for formation of a complex between the antibody or portion thereof and the target xenobiotics. The formation of a complex is then detected, wherein a difference in complex formation between the sample compared to the control sample is indicative of the presence of the target xenobiotic in the sample.

Further, within the scope of the invention is the use of compositions containing two or more xenobiotics, each xenobiotic designed to illuminate the performance of a particular organ or organ system.

Also within the scope of the invention are kits comprising the compositions (e.g., xenobiotics, antibodies or antibody fragments or derivatives such as immunoconjugates and bispecific molecules) of the invention and instructions for use. The kit can further contain a least one additional reagent, or one or more additional antibodies of the invention (e.g., an antibody having a complementary activity which binds to an epitope on a xenobiotic that is distinct from the first antibody). Kits typically include a label indicating the intended use of the contents of the kit. The term label includes any writing, or recorded material supplied on or with the kit, or which otherwise accompanies the kit.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### EXAMPLES

### Example 1

### Preparation of Iohexol

A stock solution of iohexol (10 mg/ml), commercially available from GE Healthcare as Omnipaque™, was prepared by dissolving 100 mg in 10 ml of phosphate buffered saline, pH 7.4 (diluent) containing either bovine serum albumin or human serum albumin each at a concentration of 50 mg/ml. This initial solution was serially diluted by a series of stepwise dilutions by mixing 1 part of the solution with 2 parts of diluent to a final concentration of iohexol to about 1 pg/ml.

### Example 2

### Preparation of Iodohippurate

A set of standards using sodium iodohippurate, commercially available from Sigma Aldrich (Catalog No. 10657-7) was prepared as described in Example 1 except that sodium iodohippurate was substituted for iohexol.

### Example 3

### Preparation of a Solution Containing Iohexol and Iodohippurate

A stock solution of iohexol (10 mg/ml) and sodium iodohippurate (10 mg/ml) was prepared by dissolving 100 mg of each xenobiotic in 10 ml of phosphate buffered saline (pH 7.4) (diluent) containing either bovine serum albumin or human serum albumin each at a concentration of 50 mg/ml. This solution was serially diluted 1 to 3 as described in Example 1 to a final concentration of iohexol to about 1 pg/ml.

### Example 4

### Preparation of Gadolinium-DTPA

A set of standards using gadolinium DTPA (BioPhysics Assay Laboratory, Catalog No. MR-00P10) was prepared as described in Example 1 except that gadolinium DTPA was substituted for iohexol.

### Example 5

### Preparation of Gadolinium-DOTA

A set of standards using gadolinium DOTA (commercially available from Guerbet, France) was prepared as described in example 1 except that gadolinium DOTA was substituted for iohexol.

### Example 6

### Preparation of an Iohexol-Albumin Antigen

Iohexol (140 mg) was dissolved in 1 ml of dimethylsulfoxide. To this solution was added solid dimethylaminopyridine (24 mg) and 4-nitrophenylchloroformate (30 mg). The solution was incubated at room temperature for 60 minutes with gentle mixing. The solution containing the activated iohexol was then added dropwise to a 10 ml solution of bovine albumin (10 mg/ml) dissolved in 0.2 M sodium carbonate pH 8.9. The solution was stirred for 20 hours and dialyzed exhaustively against a 12 K molecular weight cut-off (MWCO) membrane. The iodine content of the antigen was measured using neutron activation analysis (BioPhysics Assay Laboratory) and a final ratio of moles of hapten to mole of protein of 5.3 was determined.

### Example 7

### Preparation of an Iodohippurate-Albumin Antigen

Iodohippurate (230 mg) was dissolved in 5 ml of dimethylformamide. To this solution was added 105 gl of triethylamine followed by 70 μl of ethylchloroformate. The solution was incubated at room temperature for 10 minutes with gentle mixing. The solution containing the chemically activated iodohippurate was then added dropwise to a 25 ml solution of bovine albumin (20 mg/ml) dissolved in water and adjusted to pH 9.5. The solution was stirred for 2 hours and dialyzed exhaustively against a 12 K MWCO membrane. The iodine content of the antigen was measured using neutron activation analysis (BioPhysics Assay Laboratory) and a final ratio of moles of hapten to mole of protein of 38 was determined.

### Example 8

### Preparation of a Gd-DTPA-Albumin Antigen

Solid DTPA-anhydride (4.5 g) was added in small portions to bovine albumin dissolved in 120 ml of distilled water while maintaining a pH of 9.3. The resulting modified protein was exhaustively dialyzed to remove unreacted DTPA. To this purified protein was added 1.3 ml of 1 M GdCl₃ dropwise while maintaining a pH of about 7.5. The product was again exhaustively dialyzed and adjusted to a final concentration of 1 mg/ml protein. The conjugated albumin was filtered through a 0.45 micron filter. The gadolinium content of the antigen was measured using neutron activation analysis (BioPhysics Assay Laboratory) and a final ratio of moles of hapten to mole of protein of 18 was determined.

### Example 9

### Preparation of a Mercaptoacetyltriglycine (MAG₃)-albumin Conjugate

Bz-MAG₃ (300 mg) is dissolved in 50 ml of acetonitrile-water (6:4). Stannous chloride (616 mg) in 50 ml of 0.1 M citrate buffer (pH 5) and KReO₄ in 50 ml of water are added to the Bz-MAG₃. The reaction mixture is stirred and refluxed for 1 hour. After the reaction mixture has cooled to room temperature, Re-MAG₃ is purified by RP-HPLC performed with a Cosmosil 5C18-AR300 column at a flow rate of 12 ml/min with 0.1% TFA to 30% acetonitrile in water with 0.1% TFA for 30 min.

An amount of 10 mg of the isolated fraction is dissolved in MES buffer pH 6 containing 100 mg of bovine albumin. Three additions of solid water soluble carbodiimide (EDAC) (5 mg) are added over a 2 hour period. The reaction is stopped after 4 hours. The product is exhaustively dialyzed and adjusted to a final concentration of 1 mg/ml protein. The conjugated albumin is filtered through a 0.45 micron filter.

### Example 10

### Preparation of an Inulin-Albumin Conjugate

Solid inulin (1.0 g) is suspended in acetone. To this solution add solid dimethylaminopyridine (24 mg) and of 4-nitrophenylchloroformate (30 mg). The solution is incubated at room temperature for 60 minutes with gentle mixing. The solution containing the activated inulin is then added dropwise to a 100 ml solution of bovine albumin (10 mg/ml) dissolved in 0.2 M sodium carbonate pH 8.9. The solution is stirred for 20 hours. The product is again exhaustively dialyzed against a 12 K MWCO membrane and adjusted to a final concentration of 1 mg/ml protein. Filter the conjugated albumin through a 0.45 micron filter.

### Example 11

### Preparation of HRP-Conjugates

HRP was conjugated with various xenobiotics following the procedures described in Examples 6-8 except that the amount of excess of activated hapten was lowered to an amount of 1 to 20 fold molar excess over HRP. The HRP conjugate was exhaustively dialyzed against phosphate buffered saline, pH 7.4 and stored at 4° C. Similar conjugates of alkaline phosphatase and β-galactosidase can be made following similar procedures.

### Example 12

### Preparation of Polyclonal Antibodies (General Procedure)

White New Zealand rabbits were injected subcutaneously with 1 mg of antigen (1 mg/ml) in multiple sites on the rabbit's back according to the schedule in Table 2. The initial injection was prepared in Freund's complete adjuvant. All other injections were prepared in Freund's incomplete adjuvant. Test bleeds and production bleed dates are also shown in the table.

TABLE 2
Schedule for antigen injections

| | DATE | AMOUNT |
|---|---|---|
| INJECTIONS: | Apr. 21, 2005 | Inject 1 mg CFA |
| | May 12, 2005 | Inject 1 mg IFA |
| | Jun. 2, 2005 | Inject 1 mg IFA |
| | Jun. 23, 2005 | Inject 1 mg IFA |
| | Jul. 14, 2005 | Inject 1 mg IFA |
| | Aug. 11, 2005 | Inject 1 mg IFA |
| | Sep. 8, 2005 | Inject 1 mg IFA |
| | Oct. 6, 2005 | Inject 1 mg IFA |
| BLEEDS: | Apr. 21, 2005 | Pre-bleed |
| | Jun. 13, 2005 | Test Bleed |
| | Jul. 5, 2005 | Production Bleed |
| | Jul. 21, 2005 | Production Bleed |
| | Jul. 28, 2005 | Production Bleed |
| | Aug. 4, 2005 | Production Bleed |
| | Aug. 18, 2005 | Production Bleed |
| | Aug. 25, 2005 | Production Bleed |
| | Sep. 1, 2005 | Production Bleed |
| | Sep. 15, 2005 | Production Bleed |
| | Sep. 22, 2005 | Production Bleed |
| | Sep. 29, 2005 | Production Bleed |
| | Oct. 13, 2005 | Production Bleed |
| | Oct. 20, 2005 | Production Bleed |
| | Oct. 27, 2005 | Production Bleed |

### Example 13

### Preparation of a Reagent Set for Use in Immunoassay

Standard solutions. Standards were prepared for each of the xenobiotics iohexol, iodohippurate, and gadolinium-DTPA as described in Examples 1, 2 and 4. An example of a standard containing two xenobiotics is described in Example 3. Standards for other immunoassays can be prepared as described in Examples 1-5 by substituting the requisite standard for one of the standards used in Examples 1-5.

Coating polystyrene microtiter plates. Microtiter strips were coated with 200 ul of 10 ug/ml antibody solution diluted in 0.1 M carbonate buffer pH 9 and incubated overnight at room temperature. The coated plates were blocked with a phosphate buffered saline, pH 7.4 solution containing bovine serum (5 mg/ml) and dextrose 20 mg/ml) for 30 minutes, blotted dry and stored at 4° C. until used. All plates were used within 5 days of preparation. In a typical preparation the coating antibody was a goat anti-rabbit serum.

HRP-conjugates. HRP-conjugates prepared as described in Example 11 were diluted between 1 to 100 and 1 to 1000 into phosphate buffered saline, pH 7.4 buffer containing bovine albumin (1 mg/ml).

Substrate solution. HRP substrate was purchased from BioFX (Catalog TMBW-01004-01).

Stop solution. Stop reagent was purchased from BioFX (Catalog STPR-0100-01).

### Example 14

### Immunoassay Protocol

General immunoassay protocols for analytes were performed as follows. 100 μl of standard or serum sample was pipeted into appropriate wells in duplicate followed by 100 μl of antibody. To each well was added 200 μl of HRP-conjugate. After a 60 minute incubation at room temperature the wells were aspirated and washed three times with phosphate buffered saline, pH 7.4 containing (0.1% serum albumin and 0.01% Tween 20. To each well was added 200 ul of substrate solution and incubated for 30 minutes at room temperature. The development of color was stopped by the addition of 50 μl of stop solution. The wells were read using a microtiter plate reader. Standard curve data for gadolinium DTPA is shown in Table 3. Standard values were chosen specifically for this assay as shown in Table 3.

TABLE 3
Standard curve data for an assay of gadolinium DTPA

| Gadolinium DTPA, concentration M | Optical density, 450 nm |
|---|---|
| 0 | 0.69 |
| 1 × 10⁻⁶ | 0.58 |
| 5 × 10⁻⁶ | 0.61 |
| 1 × 10⁻⁵ | 0.57 |
| 5 × 10⁻⁵ | 0.55 |
| 1 × 10⁻⁴ | 0.48 |
| 1 × 10⁻³ | 0.42 |
| 1 × 10⁻² | 0.29 |

### Example 15

### Immunoassay of Iohexol

Standard curve data for iohexol is shown using the protocol described in Example 14 except that standard values are as shown in Table 4.

TABLE 4
Standard curve data for an assay of iohexol.

| Iohexol, concentration M | Optical density, 450 nm |
|---|---|
| 0 | 1.82 |
| 1 × 10−6 | 1.63 |
| 5 × 10−6 | 1.23 |
| 1 × 10−5 | 0.90 |
| 5 × 10−5 | 0.55 |
| 1 × 10−4 | 0.21 |
| 1 × 10−3 | 0.09 |
| 1 × 10−2 | 0.06 |

### Example 16

### Immunoassay of Iodohippurate

Standard or serum sample (100 μl) was pipeted into appropriate wells in duplicate followed by 100 μl of antibody. To each well was added 200 μl of HRP-iodohippurate conjugate. After a 60 minute incubation at room temperature the wells were aspirated and washed three times with phosphate buffered saline, pH 7.4 containing (0.1% serum albumin and 0.01% Tween 20. To each well was added 200 ul of substrate solution and incubated for 30 minutes at room temperature. The development of color was stopped by the addition of 50 μl of stop solution. The wells were read using a microtiter plate reader. Standard curve data for iodohippurate are shown in Table 5. Standard values were chosen specifically for this assay as shown in Table 5.

TABLE 5
Standard curve data for an assay of iodohippurate

| Iodohippurate, concentration M | Optical density, 450 nm |
|---|---|
| 0 | 1.15 |
| 1 × 10⁻⁸ | 1.14 |
| 1 × 10⁻⁷ | 1.02 |
| 1 × 10⁻⁶ | 0.74 |
| 1 × 10⁻⁵ | 0.55 |
| 1 × 10⁻⁴ | 0.49 |

### Example 17

### A Comparison of Methods to Measure the Glomerular Filtration Rate of the Kidney in Humans

The goal of the example is to compare the glomerular filtration rate (GFR) as measured by three different techniques. The first technique is a standard radioactive method using a radioactive tracer (^{99m}Tc-DTPA) to measure the rate of clearance of the tracer from the body via the glomerulus. The second method uses a nonradioactive tracer (iohexol) and its clearance rate from the body via the glomerulus was measured by neutron activation analysis, as previously described (J Lab Clin Med 2003; 141:106-9). The third method also uses the nonradioactive tracer iohexol; however, its clearance rate is measured by a functional immunoassay technique.

Patients with known renal dysfunction are enrolled in the study. At the start of the study, patients are given fluids and then asked to completely void their bladders. The time of urine collection and volume are recorded. An intravenous catheter is then placed in one arm and a 5 ml blood sample is collected and the time recorded. Following collection, a second intravenous catheter is placed in the other arm. A co-injection of ^{99m}Tc-DTPA (˜1 mCi) and iohexol (5 ml, 300 mg/I per ml) are given and the time of injection recorded. Following the injection, the injection catheter is removed. The patients are encouraged to drink fluids throughout the duration of the study.

One-hour post injection, a 5 ml blood sample is collected and the time of collection is recorded. In addition, each of the patients is asked to completely void his or her bladder and the time and total volume of urine collection is recorded. Thereafter, a blood sample is collected every half hour for the next three hours. The accurate time of blood collection is recorded. In addition, a complete urine collection is obtained every hour for the next three hours. The accurate time and total volume of urine collected is recorded. The total time for the GFR test is approximately 4 hours.

From the blood samples, 1 ml of plasma from each time point is reserved for radiation counting of ^{99m}Tc-DTPA activity, and two 100 μl samples of plasma from each time point are reserved for neutron activation analysis and for functional immunoassay measurement of iohexol. Likewise, 1 ml of urine from each time point is also reserved for radiation counting of ^{99m}Tc-DTPA activity, and two 100 μl samples of urine from each time point are reserved for neutron activation analysis and for functional immunoassay measurement of iohexol.

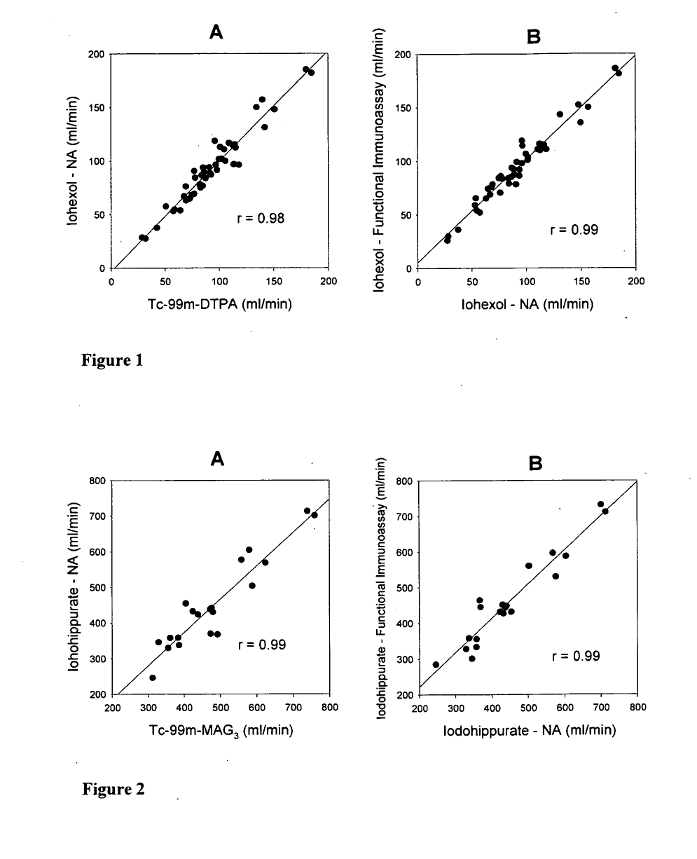

From the measured concentration of the filtration markers, the GFR value is calculated based on the kinetic clearance of the marker from the plasma and the kinetic accumulation in urine (UV/P) following standard procedures known to those skilled in the art. The results of the example are provided in FIG. 1. The data demonstrate that functional immunoassay provides an equivalent GFR value as compared to the standard radioactive method and the neutron activation method.

### Example 18

### A Comparison of Methods to Measure the Effective Renal Blood Flow of the Kidney in Humans

The goal of the example is to compare the effective renal blood flow (ERBF) as measured by two different techniques. The first technique is a standard radioactive method using a radioactive tracer (^{99m}Tc-MAG₃) to measure the rate of clearance of the tracer from the body via tubular excretion from the kidney. The second method uses the nonradioactive tracer iodohippurate; the clearance rate of this xenobiotic is measured by a functional immunoassay technique as described herein.

Twenty healthy volunteers with no history of renal dysfunction are enrolled in the study. At the start of the study, patients are given fluids and then asked to completely void their bladders. The time of urine collection and volume is recorded. An intravenous catheter is then placed in one arm and a 5 ml blood sample is collected and the time recorded. Following collection, a second intravenous catheter is placed in the other arm. A co-injection of ^{99m}Tc-MAG₃ (˜1 mCi) and iodohippurate (5 ml, 300 mg/I per ml) is given and the time of injection is recorded. Following the injection, the injection catheter is removed. The patients are encouraged to drink fluids throughout the duration of the example.

One-hour post injection, a 5 ml blood sample is collected and the time of collection is recorded. In addition, the patients are asked to completely void their bladder and the time and total volume of urine collection is recorded. Thereafter, a blood sample is collected every half hour for the next three hours. The accurate time of blood collection is recorded. In addition, thereafter, a complete urine collection is obtained every hour for the next three hours. The accurate time and total volume of urine collected is recorded. As a result, the total time for the ERBF test is approximately 4 hours.

From the blood samples, 1 ml of plasma from each time point is reserved for radiation counting of ^{99m}Tc-MAG₃ activity, and a 100 μl sample of plasma from each time point is reserved for functional immunoassay measurement of iodohippurate. Likewise, 1 ml of urine from each time point is also reserved for radiation counting for ⁹⁹mTc-MAG₃ activity, and a 100 μl sample of urine from each time point is reserved for functional immunoassay measurement of iodohippurate.

From the measured concentration of the markers in collected samples, the ERBF value is calculated based on the kinetic clearance of the marker from the plasma and the kinetic accumulation in urine (ΔUV/ΔP). Expected results of the example are provided in FIG. 2. The data demonstrate that functional immunoassay provides a comparable ERBF value as compared to the standard radioactive method.

### Example 19

### A Comparison of Methods to Measure the Filtration Fraction of the Kidney in Humans

The goal of the example is to (1) compare the filtration fraction (FF) of the kidney as measured by two different techniques and to (2) demonstration that functional immunoassay technology can measure more than one functional parameter simultaneously. The first technique is a standard radioactive method using two different radioactive tracers. The first radioactive tracer is ¹²⁵I-iothalamate to measure GFR and the second radioactive tracer is ^{99m}Tc-MAG₃ to measure ERBF. The second technique uses two nonradioactive tracers. The first nonradioactive tracer is iohexol for the measurement of GFR and the second nonradioactive tracer is iodohippurate for the measurement of ERBF. The clearance rate of these nonradioactive tracers is measured by a functional immunoassay technique.

Twenty healthy volunteers with no history of renal dysfunction are enrolled in the study. At the start of the study, patients are given fluids and then asked to completely void their bladders. The time of urine collection and volume are recorded. An intravenous catheter is then placed in one arm of each volunteer and a 5 ml blood sample is collected and the time recorded. Following collection, a second intravenous catheter is placed in the other arm. A co-injection of ¹²⁵I-iothalamate and ^{99m}Tc-MAG₃ is given, directly followed by a co-injection of iohexol and iodohippurate. The time of injections is recorded. Following the injections, the injection catheter is removed. The patients are encouraged to drink fluids throughout the duration of the study.

One-hour post injection, a 5 ml blood sample is collected and the time of collection is recorded. In addition, the patients are asked to completely void their bladder and the time and total volume of urine collection is recorded. Thereafter, a blood sample is collected every half hour for the next three hours. The accurate time of blood collection is recorded. In addition a complete urine collection is obtained every hour for the next three hours. The accurate time and total volume of urine collected is recorded. As a result, the total time for the duel ERBF tests is approximately 4 hours.

From the blood samples, 1 ml of plasma from each time point is reserved for radiation counting for ¹²⁵I-iothalamate and ^{99m}Tc-MAG₃ activity, and a 100 μl sample of plasma from each time point is reserved for functional immunoassay measurement of iohexol and iodohippurate. Likewise, 1 ml of urine from each time point is also reserved for radiation counting, and a 100 μl sample of urine from each time point is reserved for functional immunoassay measurement.

From the measured concentration of the markers in collected samples, the GFR and the ERBF values are calculated based on the kinetic clearance of each marker from the plasma and the kinetic accumulation of each marker in urine (UV/P). Given the hematocrit, the effective renal plasma flow (ERPF) is calculation, as follows

ERPF=(1−Hct)A ERBF.

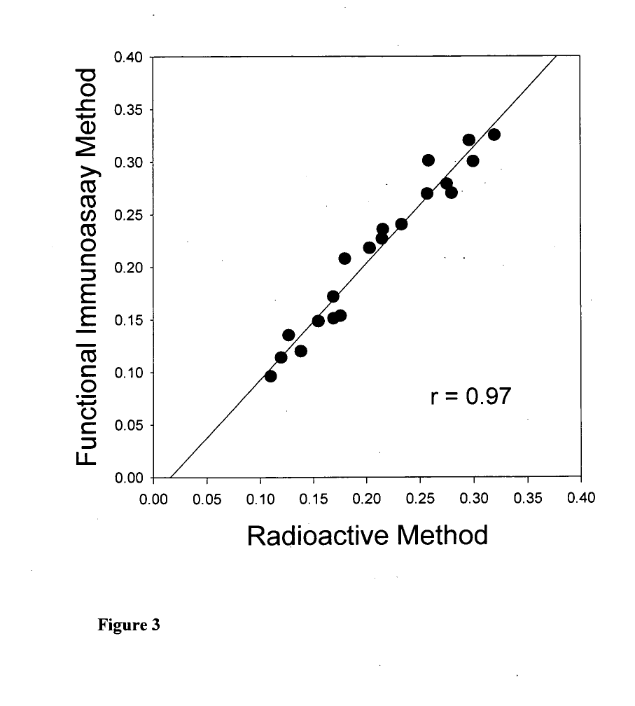

As a result, the FF is calculated from the ratio of the GFR and the ERPF, i.e., (FF=GFR/ERPF). Expected results of the example are provided in FIG. 3. The data demonstrate that functional immunoassay provides a comparable FF value as compared to the standard radioactive method and that the functional immunoassay techniques can effectively measure more than one functional parameter simultaneously.

### Example 20

### A Comparison of Methods to Measure the Plasma Volume of the Circulatory System in Humans

The goal of the example is to compare the plasma volume as measured by two different techniques. The first technique is a standard radioactive method using a radioactive tracer (¹²⁵I-human serum albumin) to measure the plasma volume of the circulatory system of the body. The second method uses the nonradioactive tracer (dextran), wherein the plasma volume of the circulatory system of the body is measured by a functional immunoassay technique.

Twenty healthy volunteers are enrolled in the study (10 male and 10 female). Patients are rested for 15 minutes prior to the start of the study. Each patient is then administered a co-injection of ¹²⁵I-human serum albumin (˜10 CCi) and dextran (1 mg), intravenously. Using a collection site different from the injection site, heparinized blood samples are collected at 10, 20, and 30 minutes post injection.

From the blood samples, 1 ml of plasma from each time point is reserved for radiation counting for ¹²⁵I-human serum albumin activity, and a 100 μl sample of plasma from each time point is reserved for functional immunoassay measurement of dextran.

From the measured concentration of the markers in collected samples, the plasma value is calculated based on the kinetic clearance of the marker. The measured activity is plotted against time on semilogarithmic paper. The best straight line is drawn through the points. The zero-time activity for each tracer is estimated by extrapolation and is used for the calculation of the plasma volume. The plasma volume (ml) is calculated by the activity concentration of the injection divided by the activity of the plasma sample obtained by extrapolation. The data demonstrate that functional immunoassay provides a comparable measurement of the plasma volume as compared to the standard radioactive method.

### Example 21

### A Comparison Study Designed to Measure the Binding Capacity of the Asialoglycoprotein of the Hepatic System in Normal and Diabetic Rats

The goals of the example are (1) to compare the binding capacity of the asialoglycoprotein receptor in normal and diabetic rats, and (2) to compare two different methods of obtaining a measure of performance of this hepatic functional parameter. The traditional radioactive tracer method will be directly compared to the functional immunoassay technique.

Diabetes is induced in 10 rats by treatment with streptozotocin. Ten untreated rats serve as a control group. Each diabetic rat is administered a co-injection of 100 μCi of ¹²⁵I-labeled asialoglycoprotein and 100 μg of bovine asialoglycoprotein, intravenously. Using a collection port different from the injection site, one blood sample (50 μl) is drawn 30 seconds post injection and then every 15-minutes for two hours. The concentration of radiolabeled asialoglycoprotein is measured and the concentration of non-rat asialoglycoprotein is measure via a functional immunoassay technique. The activity concentration for all the blood samples is normalized to the first blood sample. The normalized activity is plotted against time on semilogarithmic paper and the kinetic clearance rate (t_{1/2}) is calculated. The data obtained demonstrate that functional immunoassay provides a comparable t_{1/2} value for the asialoglycoprotein receptor response as compared to the standard radioactive method.

What is claimed is:

1. A composition comprising a first antibody selected from the group comprising anti-iohexol, anti-iothalamate, anti-ioversol, anti-gadolinium-DTPA, anti-gadolinium-DOTA, anti-GdHP-D03A, anti-chromium-EDTA, and anti-inulin; and
a second antibody selected from the group comprising anti-para-aminohippuric acid, anti-iodohippurate and anti-mercaptoacetyltriglycine-rhenium (MAG₃-Re).

2. The composition according to claim 1, wherein a first immunoassay comprising the first antibody measures glomerular filtration rate and a second immunoassay comprising the second antibody measures effective renal blood flow, and wherein the composition is a reagent for the first and the second immunoassay performed together in a single tube.

3. A composition for determining glomerular filtration rate or effective renal blood flow, wherein the composition comprises at least one antibody that binds at least one xenobiotic selected from the group iohexol, iothalamate, ioversol, gadolinium-DTPA, gadolinium-DOTA, GdHP-D03A, chromium-EDTA, inulin, para-aminohippuric acid, iodohippurate, and mercaptoacetyltriglycine-rhenium (MAG₃-Re), wherein determining glomerular filtrations rate or effective renal blood flow is performing an immunoassay with the antibody, thereby determining a concentration of the xenobiotic in at least one post-administration sample from a subject.

4. The composition according to claim 3 wherein the antibody comprises an antibody fragment.

5. The composition according to claim 3 wherein the antibody is polyclonal.

6. The composition according to claim 3 wherein the antibody is monoclonal.

7. The composition according to claim 3 wherein the antibody is formulated to perform immunoassay.

8. The composition according to claim 3 wherein the antibody is an antiserum.

9. The composition according to claim 3 wherein the antibody is a solid.

10. The composition according to claim 3 wherein the antibody is in solution.

11. The composition according to claim 3 wherein the antibody is attached to a solid phase.

12. An immunizing composition comprising an antigen for producing antibodies to a xenobiotic selected from the group consisting of iohexol, iothalamate, ioversol, GdHP-D03A, chromium-EDTA, inulin, para-aminohippuric acid, iodohippurate, and mercaptoacetyltriglycine-rhenium (MAG₃-Re).

13. A diagnostic kit for quantifying by functional immunoassay a physiological process selected from at least one of glomerular filtration rate, effective renal blood flow, filtration fraction, blood volume, intravascular space, and bile acid activity, the kit comprising a xenobiotic composition, an antibody for the xenobiotic, a detection probe, a container, and instructions for use.

14. A composition comprising a first xenobiotic selected from the group comprising iohexol, iothalamate, ioversol, gadolinium-DTPA, gadolinium-DOTA, GdHP-D03A, chromium-EDTA, and inulin and a second xenobiotic selected from the group comprising para-aminohippuric acid, iodohippurate and mercaptoacetyltriglycine-rhenium (MAG₃-Re).

15. The composition according to claim 14 wherein the composition is an immunoassay standard.

16. The composition according to claim 14 wherein the composition is a pharmaceutical.

17. A method for quantifying at least one physiological process, the method comprising:
obtaining at least one physiological material from a subject, wherein the subject is further administered a composition comprising at least one xenobiotic, and obtaining between 1 minute and 5 days following administration of the xenobiotic at least one post-administration sample of the physiological material from the subject;
determining a concentration of the xenobiotic in the at least one sample by at least one immunoassay technique; and
calculating a rate of change from a rate formula of the xenobiotic in the post-administration sample of the physiological material and expressing quantitatively the performance of the physiological process from the rate data.

18. The method according to claim 17 wherein the rate formula conforms to a one compartmental model.

19. The method according to claim 18, wherein the one compartmental model is mathematically expressed as
C₁(t)=C₁(0)e^{-kt}
wherein C₁ is the concentration of the xenobioticin the compartment at a point in time, t, and k is the rate of the xenobiotic leaving or entering the compartment.

20. The method according to claim 17 wherein the rate formula conforms to an at least one compartmental model.

21. The method according to claim 17 wherein the physiological material is at least one sample selected from the group comprising blood, urine, cerebrospinal fluid, lymph, tissue, and feces.

22. The method according to claim 17 wherein the xenobiotic is at least one xenobiotic selected from the group comprising iohexol, iothalamate, ioversol, gadolinium-DTPA, gadolinium-DOTA, GdHP-D03A, chromium-EDTA, inulin, para-aminohippuric acid, iodohippurate, mercaptoacetyltriglycine-rhenium, colloids, dextran, dextran derivatives, bile acid derivatives and analogs, and albumin derivatives.

23. The method according to claim 22 wherein a diameter of a xenobiotic colloidal particle is in the range of 2 nanometers to 1000 nanometers.

24. The method according to claim 17 wherein the physiological process measures kidney function.

25. The method according to claim 24 wherein the kidney function is selected from the group of glomerular filtration rate, effective renal blood flow, filtration fraction, and glomerular sieving.

26. The method according to claim 17 wherein the xenobiotic is a glomerular filtration rate marker.

27. The method according to claim 26 wherein the glomerular filtration rate marker is selected from the group consisting of iohexol, iothalamate, ioversol, gadolinium-DTPA, gadolinium-DOTA, GdHP-D03A, chromium-EDTA, and inulin.

28. The method according to claim 17 wherein the xenobiotic is excreted through the glomeruli of the kidney.

29. The method according to claim 17 wherein the xenobiotic is an effective renal blood flow marker.

30. The method according to claim 29 wherein the effective renal blood flow marker is selected from the group comprising para-aminohippuric acid, iodohippurate and mercaptoacetyltriglycine-rhenium.

31. The method according to claim 17 wherein the xenobiotic is excreted through the tubules of the kidney.

32. The method according to claim 17 comprising formulating the composition to contain a first xenobiotic for measuring the glomerular filtration rate of the kidney and a second xenobiotic for measuring the effective renal blood flow of the kidney.

33. The method according to claim 32 wherein the first xenobiotic is selected from the group of glomerular filtration rate markers consisting of iohexol, iothalamate, ioversol, gadolinium-DTPA, gadolinium-DOTA, GdHP-D03A, chromium-EDTA, and inulin, and the second xenobiotic is selected from the group of effective renal blood flow markers consisting of para-aminohippuric acid, iodohippurate and mercaptoacetyltriglycine-rhenium.

34. The method according to claim 17 wherein the composition comprises at least one marker of glomerular filtration, wherein the marker of glomerular filtration is excreted through the glomeruli of the kidney, and comprises at least one marker of effective renal blood flow, wherein the marker of effective renal blood flow is excreted through the tubules of the kidney.

35. The method according to claim 17 wherein the physiological process is the glomerular filtration rate.

36. The method according to claim 35 wherein the glomerular filtration rate is expressed in ml/minutes and is calculated from the formula
glomerular filtration rate=UV/P
wherein U is the concentration of the glomerular filtration marker in urine having units of mg/ml, V is flow rate of urine formation expressed in units of ml/min and P is concentration of the glomerular filtration marker measured in blood or plasma sample and expressed in units of mg/ml.

37. The method according to claim 35 further comprising quantifying the glomerular filtration rate from the concentration of the xenobiotic in at least one blood, serum or plasma sample that was obtained at one or more measured time points.

38. The method according to claim 35 further comprising quantifying the glomerular filtration rate from the concentration of the xenobiotic in at least one blood, serum or plasma sample and at least one urine sample obtained at one or more measured time points.

39. The method according to claim 35 further comprising quantifying the glomerular filtration rate from the concentration of the xenobiotic in at least one urine sample obtained at one or more measured time points.

40. The method according to claim 17 wherein the at least one physiological process is effective renal blood flow.

41. The method according to claim 40 wherein the effective renal blood flow is expressed in ml/minutes and is calculated from the formula
effective renal blood flow=UV/P
where U is the concentration of the effective renal blood flow marker measured in urine having units of mg/ml, V is the flow rate of urine formation expressed in units of ml/min and P is the concentration of the effective renal blood flow marker measured in the blood or plasma sample and expressed in units of mg/ml.

42. The method according to claim 41 wherein quantifying the effective renal blood flow value is assaying the concentration of the xenobiotic in at least one blood, serum or plasma sample that was obtained at one or more measured time points.

43. The method according to claim 41 wherein quantifying the effective renal blood flow is assaying the concentration of the xenobiotic in at least one blood, serum or plasma sample and at least one urine sample that was obtained at one or more measured time points.

44. The method according to claim 41 wherein quantifying the effective renal blood flow assaying the concentration of the xenobiotic in at least one urine sample that was obtained at one or more measured time points.

45. The method according to claim 17, wherein the performance in the subject of the physiological process is abnormal.

46. The method according to claim 17, wherein the performance in the subject of the physiological process is normal.

47. The method according to claim 17 wherein the at least one physiological process measures a circulatory system function.

48. The method according to claim 47 wherein the physiological process of the circulatory system is selected from blood volume, plasma volume, and intravascular volume.

49. The method according to claim 17 wherein the xenobiotic is selected from the group of dextran, dextran derivatives, and derivatized albumin.

50. The method according to claim 17 wherein the administered composition comprises a first xenobiotic for measuring plasma volume of the circulatory system and a second xenobiotic for measuring intravascular volume of the circulatory system.

51. The method according to claim 50 wherein the first xenobiotic is selected from the group of plasma volume markers comprising dextran, reduced dextran, albumin; and the second xenobiotic is selected from the group of intravascular volume markers comprising colloids.

52. The method according to claim 17 wherein the physiological process comprises at least one liver function.

53. The method according to claim 52 wherein the liver function is selected from asialoglycoprotein receptor activity, reticuloendothelial system activity, bile acid absorption, and bile acid elimination.

54. The method according to claim 17 wherein the subject is a mammal.

55. The method according to claim 54 wherein the mammal is a human.

56. The method according to claim 17 wherein the subject is an isolated organ system.

57. The method according to claim 17 wherein administering the xenobiotic is administering a plurality of doses for determining sequential performance of at least one physiological process.

58. The method according to claim 17 wherein the immunoassay technique comprises using at least one reagent selected from the group of an antibody, a binding protein, and a peptide.

59. The method according to claim 17 wherein the immunoassay technique comprises at least one antibody that binds at least one xenobiotic selected from the group comprising anti-iohexol, anti-iothalamate, anti-ioversol, anti-gadolinium-DTPA, anti-gadolinium-DOTA, anti-GdHP-D03A, anti-chromium-EDTA, anti-inulin, anti-para-aminohippuric acid, anti-iodohippurate, anti-mercaptoacetyltriglycine-rhenium, anti-colloids, anti-dextran, anti-dextran derivatives, and anti-albumin derivatives.

60. The method according to claim 59 wherein the antibody comprises an antibody fragment.

61. The method according to claim 59 wherein the antibody is polyclonal.

62. The method according to claim 59 wherein the antibody is monoclonal.

63. The method according to claim 17 wherein the immunoassay technique comprises at least one binding protein or peptide that binds the xenobiotic, wherein the xenobiotic is selected from the group comprising: iohexol, iothalamate, ioversol, gadolinium-DTPA, gadolinium-DOTA, GdHP-D03A, chromium-EDTA, inulin, para-aminohippuric acid, iodohippurate, mercaptoacetyltriglycine-rhenium, colloids, dextran, dextran derivatives, and albumin derivatives.

64. The method according to claim 17 wherein the immunoassay technique comprises a first antibody that binds a first xenobiotic selected from the group comprising iohexol, iothalamate, ioversol, gadolinium-DTPA, gadolinium-DOTA, GdHP-D03A, chromium-EDTA, and inulin; and comprises a second antibody that binds a second xenobiotic selected from the group comprising para-aminohippuric acid, iodohippurate and -mercaptoacetyltriglycine-rhenium.

65. The method according to claim 17 wherein the immunoassay technique comprises a radio immunoassay, an enzyme immunoassay, an enzyme-linked immunosorbent assay, a fluorescence immunoassay, an automated immunoassay, a luminescent immunoassay and the like.

66. The method according to claim 17 wherein the xenobiotic is approved for human use.

67. The method according to claim 17 wherein the xenobiotic is approved for veterinary use.

68. A method for quantifying at least one physiological process in a subject, the method comprising:
administering to the subject at least one xenobiotic, and obtaining between 1 minute and 5 days following administration of the xenobiotic at least one post-administration sample of at least one physiological material from the subject; determining a concentration of the xenobiotic in the sample by at least one immunoassay technique; and
calculating a rate of change from a rate formula of the xenobiotic in the post-administration sample of the physiological material and expressing quantitatively the performance of the physiological process from the rate data.
